(19) United States
(12) Patent Application Publication
(10) Pub. No.: US 2016/0066796 A1
(43) Pub. Date: Mar. 10, 2016

(54) BIOLOGICAL INFORMATION MEASURING APPARATUS

(71) Applicant: UNION TOOL CO., Tokyo (JP)

(72) Inventors: Ryo SHINOZAKI, Tokyo (JP); Makoto ANEZAKI, Tokyo (JP); Katsuaki SAKATA, Tokyo (JP); Hisahiro MATSUHASHI, Tokyo (JP)

(73) Assignee: UNION TOOL CO., Tokyo (JP)

(21) Appl. No.: 14/578,825

(22) Filed: Dec. 22, 2014

(30) Foreign Application Priority Data
Sep. 9, 2014 (JP) 2014-182901

(51) Int. Cl. A61B 5/0205; A61B 5/0456; A61B 5/0472; A61B 5/00; A61B 5/04; A61B 5/0452; A61B 5/0408; A61B 5/11

(52) U.S. Cl. CPC A61B 5/02055; A61B 5/04085; A61B 5/0456; A61B 5/1121; A61B 5/0006; A61B 5/0008; A61B 5/04017; A61B 5/04525; A61B 5/0472; A61B 5/1102; A61B 5/01

(57) ABSTRACT

Provided is a practical biological information measuring apparatus that is compact, provides minimal inconvenience worn on the body, and has an extended battery life, the biological information measuring apparatus being capable of instantly displaying a heartbeat interval. A biological information measuring apparatus capable of measuring at least a heartbeat interval of a living body and capable of being attached to a living body, the biological information measuring apparatus characterized in comprising: a plurality of electrodes that come in contact with the living body; signal processing means for electrically processing a change in voltage obtained from the electrodes and creating an electrocardiogram signal; heartbeat interval measuring means for measuring, from the electrocardiogram signal, a heartbeat interval from an interval between one R wave and another adjacent R wave or an interval between one S wave and another adjacent S wave in the electrocardiogram signal; triaxial acceleration measuring means; and temperature measuring means; the biological information measuring apparatus further comprising wireless transmission means for simultaneously and wirelessly transmitting the heartbeat interval obtained by the heartbeat interval measuring means, the triaxial acceleration obtained by the triaxial acceleration measuring means, and the temperature obtained by the temperature measuring means.

### TECHNICAL FIELD

The present invention relates to a biological information measuring apparatus.

### BACKGROUND ART

The pulsation cycle of a heart (the cardiac cycle); i.e., the heartbeat interval, includes fluctuations caused by various mechanisms. Diurnal variations are an example of fluctuations of a comparatively long cycle, and it is understood that on average, the heart rate is low when one is asleep, and high when one is awake (Non-patent Document 1). There are fluctuations in short cycles as well. The heart rate decreases when one exhales and increases when one inhales. Fluctuations occur in the heartbeat intervals when these actions are repeated. For example, if the respiration rate is 15 breaths per 60 seconds, the equivalent 15/60=0.25 Hz is the fluctuation frequency.

The heart rate is managed by sympathetic nerves and vagus nerves (parasympathetic nerves). The adjusting of heartbeat intervals by the sympathetic nerves and the vagus nerves involves a transfer function; specifically, the sympathetic nerves do not transfer heartbeat interval variations of frequencies 0.15 Hz or greater, while vagus nerves transfer heartbeat interval variations up to 1 Hz (Non-patent Document 2). Because of this function, when the frequency spectrum of the heartbeat interval variations is obtained, two components are observed: a low-frequency component of 0.04 Hz or greater and less than 0.15 Hz, and a high-frequency component of 0.15 Hz or greater (Non-patent Document 3). Referring to these components as LF and HF respectively, HF is thought to reflect activity of vagus nerves, and LF is thought to reflect activity of both sympathetic nerves and vagus nerves, for the reasons described above. Furthermore, according to Maliani et al., LF/HF represents an autonomic nerve balance (Non-patent Document 4).

When the sympathetic nerves are aggravated, the heart rate increases and both LF and LF/HF increase. This indicates a state of excitement. When the vagus nerves are aggravated, the heart rate decreases, HF increases, and LF/HF decreases. This indicates a relaxed state. There are very many cases of human mental analysis that use such characteristics, examples of which include emotional evaluation during TV viewing (Non-patent Document 5), driver stress evaluation (Non-patent Document 6), relaxation evaluation during music listening or video viewing (Non-patent Document 7), and the like.

A person's autonomic nerve balance or stress level can be evaluated by measuring the heartbeat interval in this manner, but in conventional practice, heartbeat interval measurements have been taken using either a stationary electrocardiograph or a Holter electrocardiograph, which attaches to a living body and stores an electrocardiogram (waveform data) of a predetermined time duration.

However, the specification of such devices far surpasses what is required merely to measure heartbeat intervals; specifically, large-capacity memory is needed for these devices to gather all the waveform data of an electrocardiogram, the battery and the main body of the electrocardiograph proportionately increase in size, the cost is higher, and the gathered waveform data must be analyzed afterward, which is troublesome.

### PRIOR ART DOCUMENTS

### Non-Patent Documents

[Non-patent Document 1] Vanoli E, Adamson P B, Ba-Lin, et al., “Heart rate variability during specific sleep stage: A comparison of healthy subjects with patients after myocardial infarction,” Circulation, 91, 1918-1922 (1995).

[Non-patent Document 2] Berger R D, Saul J P, Cohen R J, “Transfer function analysis of autonomic regulation, I: canine atrial rate response,” Am J Physiol, 256, H142-H152 (1989).

[Non-patent Document 3] Sayers B. McA., “Analysis of heart rate variability,” Ergonomics, 16, 17-32 (1973).

[Non-patent Document 4] Maliani A, Pagani M, Lombardi F, Cerutti S, “Cardiovascular neural regulation explored in the frequency domain,” Circulation, 84, 482-492 (1991).

[Non-patent Document 5] Kiyomi Sakamoto, Seiji Sakashita, et al., “Effects of TV-viewing user's emotional state on physiological-psychological response measurement,” Panasonic Technical Journal, 59, No. 1, 29-34 (2013).

[Non-patent Document 6] Kazuhisa Ogawa, et al., “Effects on driver's emotional traits and driving behavior: for the purpose of developing educational programs for controlling emotions,” International Traffic Safety Society Report (2009).

[Non-patent Document 7] Aruku Yamada, Akiko Kataoka, “Effects of preferred music and classical music on autonomic nervous system,” Human Care Research, 7, 73-81 (2006).

### DISCLOSURE OF THE INVENTION

### Problems the Invention is Intended to Solve

The present invention was devised in view of the situation described above, it being an object thereof to provide a practical biological information measuring apparatus that emphasizes heartbeat interval measurement, and is thereby compact, minimally inconvenient when worn on the body, capable of achieving extended battery life, and can display a heartbeat interval instantly.

### Means for Solving these Problems

The main points of the present invention are described.

The present invention relates to a biological information measuring apparatus capable of measuring at least a heartbeat interval of a living body and capable of being attached to a living body, the biological information measuring apparatus characterized in comprising: a plurality of electrodes that come in contact with the living body; signal processing means for electrically processing a change in voltage obtained from the electrodes and creating an electrocardiogram signal; heartbeat interval measuring means for measuring, from the electrocardiogram signal, a heartbeat interval from an interval between one R wave and another adjacent R wave or an interval between one S wave and another adjacent S wave in the electrocardiogram signal; triaxial acceleration measuring means; and temperature measuring means; wherein the biological information measuring apparatus also comprises wireless transmission means for simultaneously and wirelessly transmitting the heartbeat interval obtained by the heartbeat interval measuring means, the triaxial acceleration obtained by the triaxial acceleration measuring means, and the temperature obtained by the temperature measuring means.

The present invention also relates to a biological information measuring apparatus which is the biological information measuring apparatus according the first aspect, characterized in being configured so that a high-pass filter is provided to the signal processing means, the heartbeat interval measuring means isochronally samples the electrocardiogram signals passing through the high-pass filter, peaks pertaining to R waves or S waves are detected at two points using a method described below, and a heartbeat interval is measured using an interval between peaks pertaining to R waves or S waves either as an interval between one R wave and another adjacent R wave, or as an interval between one S wave and another adjacent S wave:
A region is retrieved where adjacent amplitudes V satisfy the relationships for a downward trough condition: V(0)<V(1)>V(2), V(n)>V(n+1)<V(n+2), and V(m)<V(m+1)>V(m+2), or the relationships for an upward peak condition: V(0)>V(1)<V(2), V(n)<V(n+1)>V(n+2), and V(m)>V(m+1)<V(m+2); and V(n+1) in the region satisfying the downward trough condition or the upward peak condition is detected as a peak pertaining to an R wave or a peak pertaining to an S wave. The V parameters represent a time series, where 0<n<m. The amplitudes V are values outside of a predetermined noise threshold.

The present invention also relates to a biological information measuring apparatus which is the biological information measuring apparatus according the first aspect, characterized in being configured so that that a high-pass filter is provided to the signal processing means, the heartbeat interval measuring means isochronally samples the electrocardiogram signals passing through the high-pass filter, peaks pertaining to R waves or S waves are detected at two points using a method described below, and a heartbeat interval is measured using an interval between peaks pertaining to R waves or S waves either as an interval between one R wave and another adjacent R wave, or as an interval between one S wave and another adjacent S wave:
(1) A region is retrieved where adjacent amplitudes V satisfy the relationships for a downward trough condition: V(0)<V(1)>V(2), V(n)>V(n+1)<V(n+2), and V(m)<V(m+1)>V(m+2), or the relationships for an upward peak condition: V(0)>V(1)<V(2), V(n)<V(n+1)>V(n+2), and V(m)>V(m+1)<V(m+2); and V(n+1) in the region satisfying the downward trough condition or the upward peak condition is detected as a peak pertaining to an R wave or a peak pertaining to an S wave. The V parameters represent a time series, where 0<n<m. The amplitudes V are values outside of a predetermined noise threshold.
(2) After any peak fulfilling the downward trough condition or the upward peak condition and pertaining to an R wave or an S wave has been detected, a peak fulfilling the same condition and pertaining to an adjacent R wave or S wave is detected.

The present invention also relates to a biological information measuring apparatus which is the biological information measuring apparatus according the second aspect, characterized in that the heartbeat interval measuring means either sets m to 0.12 seconds or less or sets m-n to 0.06 seconds or less.

The present invention also relates to a biological information measuring apparatus which is the biological information measuring apparatus according the second aspect, characterized in that the heartbeat interval measuring means is configured so that: respective wave height values {whichever is greater between |V(1)−V(n+1)| and |V(n+1)−V(m+1)|} in the retrieved regions satisfying the conditions are compared; the downward trough condition is fulfilled when a ratio Uh/Lh, which is a ratio of a wave height value Uh in the region satisfying the upward peak condition to a wave height value Lh in the region satisfying the downward trough condition, is equal to or less than a threshold Th; and the upward peak condition is fulfilled when a ratio Lh/Uh, which is a ratio of the wave height value Lh to the wave height value Uh, is equal to or less than a threshold Th.

The present invention also relates to a biological information measuring apparatus which is the biological information measuring apparatus according the fifth aspect, characterized in that the threshold Th is 0.4 to 0.7.

The present invention also relates to a biological information measuring apparatus which is the biological information measuring apparatus according to the sixth aspect, characterized in that the heartbeat interval measuring means is configured so that once a peak pertaining to either an R wave or an S wave has been detected, a peak pertaining to the next R wave or S wave is not detected within a range of 0.3 second or less.

The present invention also relates to a biological information measuring apparatus which is the biological information measuring apparatus according to the second aspect, characterized in that the high-pass filter lets through only frequencies of at least a predetermined threshold set in a range of 8 Hz to 30 Hz.

The present invention also relates to a biological information measuring apparatus which is the biological information measuring apparatus according to the eighth aspect, characterized in that a low-pass filter is provided to the signal processing means.

The present invention also relates to a biological information measuring apparatus which is the biological information measuring apparatus according the ninth aspect, characterized in that the low-pass filter lets through only frequencies at or below a predetermined threshold set to a value greater than 30 Hz.

The present invention also relates to a biological information measuring apparatus which is the biological information measuring apparatus according the second aspect, characterized in comprising wireless transmission means for grouping the heartbeat interval obtained by the heartbeat interval measuring means, the triaxial acceleration obtained by the triaxial acceleration measuring means, and the temperature obtained by the temperature measuring means as a single living body information group, and wirelessly transmitting one to ten living body information groups simultaneously to a recording device.

The present invention also relates to a biological information measuring apparatus which is the biological information measuring apparatus according the eleventh aspect, characterized in that the triaxial acceleration measuring means is configured so as to measure the maximum value or average value of the absolute values of accelerations in each of three axes either between one R wave and another adjacent R wave or between one S wave and another adjacent S wave in the electrocardiogram signal obtained by the signal processing means.

The present invention also relates to a biological information measuring apparatus which is the biological information measuring apparatus according the eleventh aspect, characterized in that the triaxial acceleration measuring means is configured so as to measure the maximum value and minimum value of accelerations in each of three axes either between one R wave and another adjacent R wave or between one S wave and another adjacent S wave in the electrocardiogram signal obtained by the signal processing means.

The present invention also relates to a biological information measuring apparatus which is the biological information measuring apparatus according the twelfth aspect, characterized in comprising recording means for internally recording heartbeat interval, triaxial acceleration, and temperature.

The present invention also relates to a biological information measuring apparatus which is the biological information measuring apparatus according the thirteenth aspect, characterized in comprising recording means for internally recording heartbeat interval, triaxial acceleration, and temperature.

### Effect of Invention

The present invention is a practical biological information measuring apparatus that, due to being configured as described above, is compact, provides minimal inconvenience when worn on the body, offers extended battery life, and allows a heartbeat interval to be instantly display.

### BRIEF DESCRIPTION OF THE DRAWINGS

FIG. 1 is a schematic diagram of the present example;

FIG. 2 is a schematic diagram illustrating the wearing method of the present example;

FIG. 3 is a schematic diagram of a common electrocardiogram;

FIG. 4 is a schematic diagram of an electrocardiogram that has gone through a high-pass filter;

FIG. 5 is a schematic diagram illustrating P waves, Q waves, R waves, S waves, and heartbeat interval;

FIG. 6 is a schematic diagram of an Other Example 1;

FIG. 7 is a schematic diagram of an electrocardiogram before being processed by the signal processing means;

FIG. 8 is a schematic diagram of a processed electrocardiogram, wherein the electrocardiogram of FIG. 7 has been processed by the signal processing means; and

FIG. 9 is an enlarged schematic diagram showing an enlargement of the QRS width portion in FIG. 8.

### BEST MODE FOR CARRYING OUT THE INVENTION

Preferred embodiments of the present invention are briefly described with reference to the diagrams while indicating the effects of the present invention.

Electrodes are brought in contact with a living body and a change in voltage obtained from the electrodes is electrically processed to create an electrocardiogram signal, and, based on the electrocardiogram signal, a heartbeat interval is measured from the interval between one R wave in the electrocardiogram signal and another adjacent R wave or the interval between one S wave and another adjacent S wave.

In addition to the heartbeat interval measuring means for measuring the heartbeat interval, the present invention also comprises triaxial acceleration measuring means and temperature measuring means, as well as wireless transmission means for simultaneously and wirelessly transmitting the heartbeat interval, the triaxial acceleration obtained by the triaxial acceleration measuring means, and the temperature obtained by the temperature measuring means. Therefore, the posture and movement of a person can be measured from not only the heartbeat interval but also the triaxial acceleration, and the environmental temperature around the person can be measured, whereby these measurement values can be useful indicators for evaluating the stress state of the person as well as their autonomic nerve balance.

Due to these measured values being transmitted simultaneously, the biological information measuring apparatus is an apparatus in which a small-capacity memory can be used, the apparatus can be reduced in size accordingly, the wearer is therefore inconvenienced to a lesser extent, the life of the battery can be extended, and the apparatus can be satisfactorily used while worn continuously for a long period of time.

For example, the heartbeat interval measuring means can isochronally sample the electrocardiogram signals passing through a high-pass filter provided to the signal processing means, use the method described above to detect peaks pertaining to R waves or S waves at two points, and use the interval therein as an interval between one R wave and another adjacent R wave or an interval between one S wave and another adjacent S wave to measure a heartbeat interval. In this case, the heartbeat interval can be measured by an extremely simple data process performed by a microcomputer or the like, and it is possible to store only the heartbeat interval measurement results without storing even waveform data of the electrocardiogram.

### Examples

Examples of the present invention are described below with reference to the diagrams.

The present example is a biological information measuring apparatus capable of measuring at least a heartbeat interval of a living body and capable of being attached to a living body, the biological information measuring apparatus characterized in comprising: a plurality of electrodes that come in contact with the living body; signal processing means for electrically processing a change in voltage obtained from the electrodes and creating an electrocardiogram signal; heartbeat interval measuring means for measuring, from the electrocardiogram signal, a heartbeat interval from an interval between one R wave and another adjacent R wave or an interval between one S wave and another adjacent S wave in the electrocardiogram signal; triaxial acceleration measuring means; and temperature measuring means; wherein the biological information measuring apparatus also comprises wireless transmission means for simultaneously and wirelessly transmitting the heartbeat interval obtained by the heartbeat interval measuring means, the triaxial acceleration obtained by the triaxial acceleration measuring means, and the temperature obtained by the temperature measuring means.

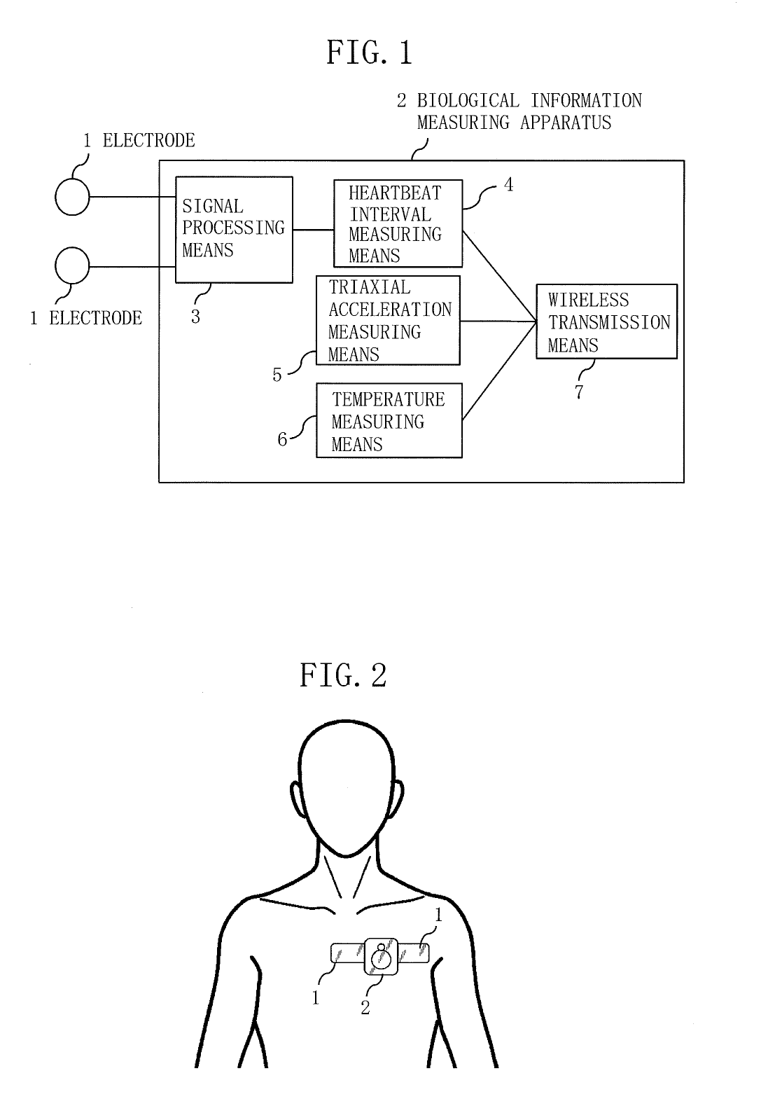

Specifically, the present example pertains to a biological information measuring apparatus 2, comprising signal processing means 3, heartbeat interval measuring means 4, triaxial acceleration measuring means 5, temperature measuring means 6, and wireless transmission means 7 as shown in FIG. 1, electrodes 1 being provided as shown in FIGS. 1 and 2. A plurality of electrodes 1 are attached to the biological information measuring apparatus 2, in which state the measurement subject attaches them to the body and the heartbeat interval and the like is measured. Three or more electrodes may be provided, but to measure the heartbeat interval, two is sufficient. Display means (a display) for displaying the measurement results is also provided in the present example.

With such a configuration in the present example, not only the heartbeat interval but also a person's posture or movement can be measured by an acceleration sensor (the triaxial acceleration measuring means 5), the environmental temperature around the person can be measured by an internal thermometer (the temperature measuring means 6), and the heartbeat interval, triaxial acceleration, and temperature can be measured in the same period without imposing a burden on the measurement subject. These measurement values can be useful indicators for evaluating the stress state of the person as well as their autonomic nerve balance, making for a more practical biological information measuring apparatus.

The present example is configured such that a high-pass filter is provided to the signal processing means 3, the heartbeat interval measuring means 4 isochronally samples the electrocardiogram signals passing through the high-pass filter, the peaks pertaining to R waves or S waves are detected at two points by a method described in detail hereinafter, and a heartbeat interval is measured using an interval between peaks pertaining to R waves or S waves either as an interval between one R wave and another adjacent R wave, or as an interval between one S wave and another adjacent S wave.

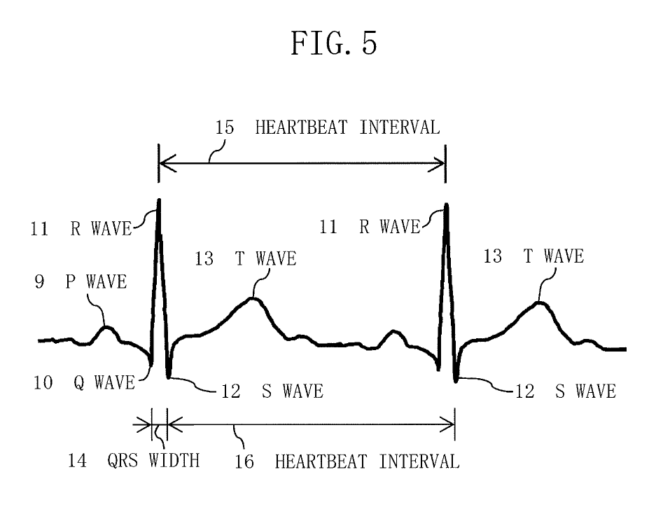

The heartbeat interval is either a temporal interval between one R wave 11 and another adjacent R wave 11 (the heartbeat interval 15), or a temporal interval between one S wave 12 and another adjacent S wave 12 (the heartbeat interval 16), as shown in FIG. 5. Specifically, when the heart's pulsation cycle for a predetermined time duration is measured, the temporal interval between one R wave 11 peaking in wave height and another R wave 11 (the adjacent R wave 11) again peaking in wave height after some passage of time is the heartbeat interval 15, and the temporal interval between one S wave 12 peaking in wave height and other S wave 12 (the adjacent S wave 12) again peaking in wave height after some passage of time is the heartbeat interval 16. This time is shown in seconds (s). Because of individual differences among measurement subjects, with a measurement subject whose R waves 11 are high, it is sufficient to measure the heartbeat interval 15 using the adjacent R wave 11, and with a measurement subject whose S waves 12 are high, it is sufficient to measure the heartbeat interval 16 using the adjacent S wave 12 (because the heartbeat intervals 15 and 16 will be the same temporal interval).

The width from a Q wave 10 in FIG. 5 to an S wave 12 is referred to as the QRS width 14, and of the wave within the QRS width 14, the R wave 11 is a wave occurring after the Q wave 10 (with some time passing after the occurrence of the Q wave 10) and having a peak in the opposite direction of the Q wave 10, and the S wave 12 represents a wave occurring after the R wave 11 (with some time passing after the occurrence of the R wave 11) and having a peak in the opposite direction of the R wave 11.

The biological information measuring apparatus 2 is made as small as possible so as to not inconvenience the measurement subject wearing the apparatus, and it is desirable that the apparatus can be operated for a long time with a small battery. In view of this, because the battery is made to have a long life in the present example, the wireless transmission means 7 is configured so as to simultaneously transmit the heartbeat interval, the triaxial acceleration, and the temperature, which are measured in the same period, as one group (living body information group) to an internal storage means 8 or an external storage device.

For example, if the heart rate is 60 bpm (60 beats per minute), a wireless transmission rate of about one transmission per second is sufficient and can contribute to reducing battery size and extending cell life. It is realistic for one to ten living body information groups to simultaneously be transmitted wirelessly to the internal storage means 8 or an external storage device, and when, for example, three living body information groups are transmitted simultaneously, there will be about one wireless communication in three seconds when the heart rate is 60 bpm, which can contribute to reducing battery size and extending cell life. It is possible for more than ten groups to be transmitted simultaneously, but the real-time quality of the data is lost regardless of the transmission being wireless and the transmission will not have the merit of instantly displaying a recognizable heartbeat interval; therefore, up to ten groups is preferred. In the present example, three living body information groups are transmitted simultaneously.

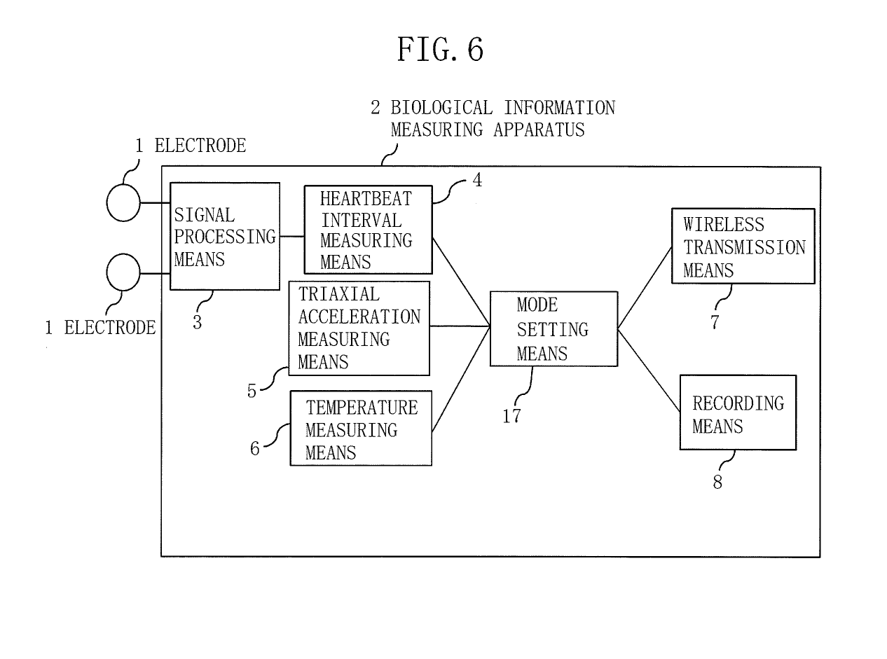

Specifically, if recording means 8 are included in addition to the wireless transmission means 7 in the biological information measuring apparatus, as in the Other Example 1 shown in FIG. 6, living body information can be recorded by a living body information measuring device alone, only the living body information groups are recorded unlike in a common Holter electrocardiograph, the information can therefore be recorded in, for example, a small semiconductor memory, the battery size is reduced, and the cell life is extended, which can contribute to reducing the size of the biological information measuring apparatus as well.

Providing the recording means 8 and mode setting means 17 in addition to the wireless transmission means 7 makes it possible to process the measured living body information groups by selecting a mode in accordance with the desired application. Specifically, the mode setting means 17 is used to select and set the mode from a transmit mode, a record mode, or a record-and-transmit mode.

When the mode is set to transmit mode, the wireless transmission means 7 can be used to transmit the measured living body information groups to an external recording device such as a personal computer, a tablet terminal, or a smartphone; when the mode is set to record mode, the internal recording means 8 can be used to record the measured living body information groups; and when the mode is set to record-and-transmit mode, the internal recording means 8 can be used to record the measured living body information groups while the wireless transmission means 7 is used to transmit the groups to an external recording device, which is more practical.

The living body information groups correspond to one heartbeat interval, but with only one, there is no perceivable change in triaxial acceleration between one R wave and another adjacent R wave or one S wave and another adjacent S wave. Therefore, a plurality of living body information groups must be confirmed in order to observe a change in triaxial acceleration. To detect, for example, a sudden change in acceleration such as when the person falls over, the triaxial acceleration measuring means 5 is preferably configured so as to measure the maximum value of the absolute values of the accelerations in all three axes between one R wave and another adjacent R wave or one S wave and another adjacent S wave. When measuring the person's posture is emphasized, there is no need to measure sudden acceleration, and the triaxial acceleration measuring means 5 is preferably configured so as to measure the average value of the accelerations in all three axes between one R wave and another adjacent R wave or one S wave and another adjacent S wave. Furthermore, to measure in detail the manner of the person's movement such as running or walking, the triaxial acceleration measuring means 5 is preferably configured so as to simultaneously measure the maximum and minimum values of the accelerations in all three axes between one R wave and another adjacent R wave or one S wave and another adjacent S wave. Another option is a configuration capable of measuring two or more groups of values among the above-described maximum value of the absolute values of the accelerations in all three axes, the average value of the accelerations in all three axes, and the maximum and minimum values of the accelerations in all three axes.

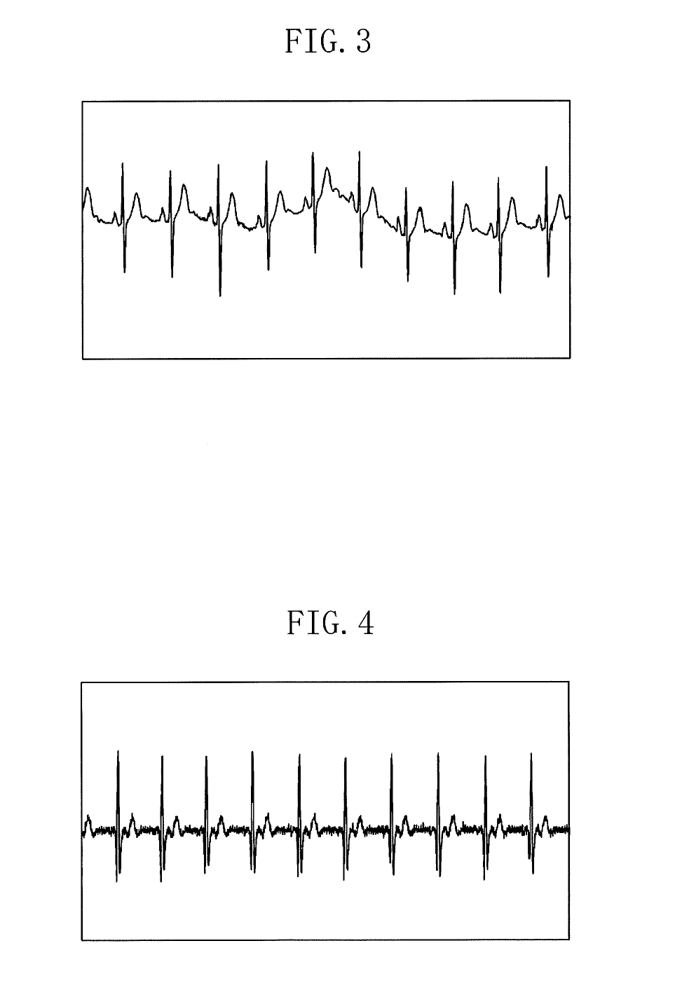

Commonly, a microcomputer is used for the heartbeat interval measuring means 4, but to distinguish between baseline fluctuation in a common electrocardiogram as shown in FIG. 3 and the T waves 13 (occurring some time after the occurrence of each S wave 12) shown in the electrocardiogram of FIG. 5, some kind of filter circuit is needed to detect the peaks pertaining to R waves or the peaks pertaining to S waves (the term “baseline” refers to a straight line that would connect the position where one P wave 9 begins to rise with the position where an adjacent P wave 9 begins to rise).

In view of this, if the filter applied as the signal processing means 3 is a high-pass filter (filter circuit) for letting through only frequencies at or above a predetermined threshold set in a range of 8 Hz to 30 Hz, e.g. a high-pass filter for letting through only frequencies of 10 Hz or more, baseline fluctuations are minimized as in FIG. 4, and peaks pertaining to R waves or S waves can be detected in a simple manner. It is preferable to let through only frequencies at or above a threshold set in a range of 8 Hz to 30 Hz because when the frequency is less than 8 Hz, baseline fluctuations are difficult to minimize, and when the frequency is greater than 30 Hz, R wave data cannot be acquired (because only components 30 Hz or less are included in the rise of an R wave and therefore the R waves cannot be confirmed when only frequencies of 30 Hz or greater are let through).

Thus, if the detection of peaks pertaining to R waves or S waves is simple, the number of steps to program the microcomputer can be reduced and power consumption is minimized, which can contribute to reducing battery size and extending cell life.

When only a high-pass filter is applied, noise such as that of myoelectricity is incorporated in the electrocardiogram, and there is a possibility that this noise will be mistakenly detected by the microcomputer as R waves or S waves. In view of this, a low-pass filter is also preferably provided to the signal processing means 3 in addition to the high-pass filter in order to reduce this possibility. Because the low-pass filter needs to let through R waves, the low-pass filter lets through only frequencies at or below a predetermined threshold set to a value greater than 30 Hz, e.g. a range from 31 Hz to 2000 Hz. A frequency greater than 2000 Hz is meaningless because there is no living body signal, and the upper limit is therefore set to 2000 Hz.

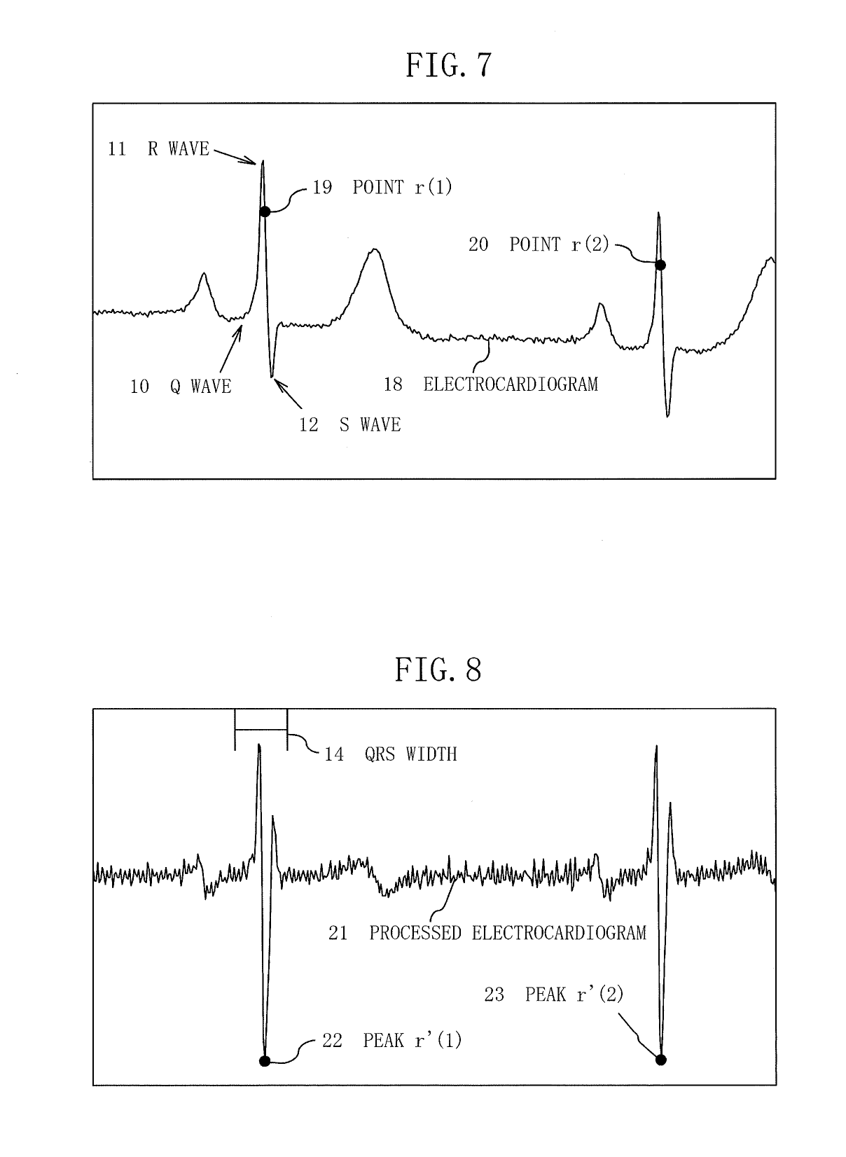

The relationship of the heartbeat interval to the peaks pertaining to R waves and the peaks pertaining to S waves will now be described. FIG. 7 is an electrocardiogram 18 obtained from the electrodes 1. When the high-pass filter of the signal processing means 3 is applied to this electrocardiogram, the processed electrocardiogram 21 of FIG. 8 is obtained. In the present example, a high-pass filter that lets through only frequencies of 15 Hz or greater is applied. What is detected by the heartbeat interval measuring means 4 is the peaks of the processed electrocardiogram 21.

The points on the electrocardiogram 18 in FIG. 7 that are concurrent with the points in FIG. 8 where the peak r′(1) 22 and the peak r′(2) 23 are measured are the point r(1) 19 and the point r(2) 20, respectively. The point r(1) 19 and the point r (2) 20 are not R wave peaks (locations where the wave peaks), but because the respective QRS waveforms of a person's electrocardiogram are similar (the QRS waveforms are the same even if the wave heights are different), the interval between adjacent peaks of the processed electrocardiogram 21, i.e. the interval between the peak r′(1) 22 and the peak r′(2) 23 is virtually equivalent to the heartbeat interval. Herein (the wave of the peak r′(1) 22 in FIG. 8 is replaced with an R wave for the sake of convenience), the peak r′(1) 22 and the peak r′(2) 23 are referred to as peaks pertaining to the R waves. If the S waves have a greater wave height than the R waves in the electrocardiogram obtained from the electrodes 1, the peak r′(1) 22 and the peak r′(2) 23 of the processed electrocardiogram 21 will be peaks pertaining to the S waves. In FIG. 8, the peak r′(1) 22 is a peaks pertaining to an R wave.

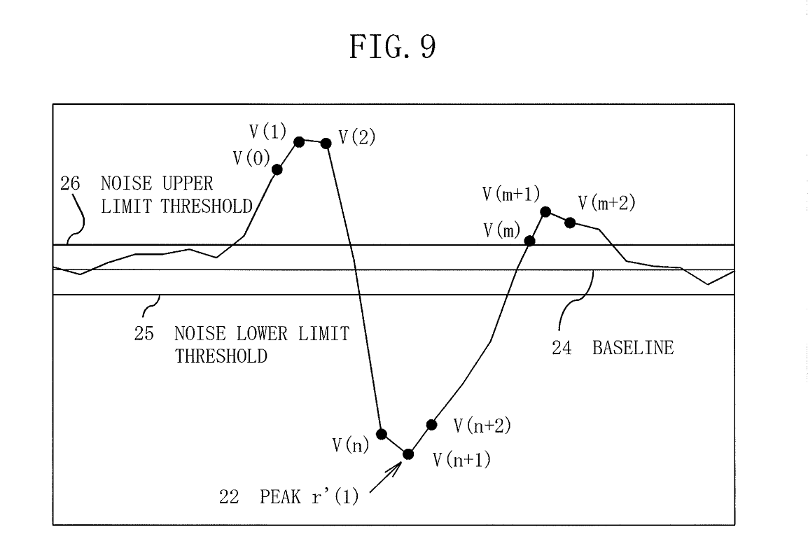

Next, the method for detecting peaks pertaining to R waves and peaks pertaining to S waves is described using FIG. 9. FIG. 9 is an enlargement of the portion of the QRS width 14 in FIG. 8. First, the processed electrocardiogram 21 to which the high-pass filter is applied is sampled at isochronal intervals, and the sampling points retrieved are adjacent amplitudes V which have the relationships V(0)<V(1)>V(2), V(n)>V(n+1)<V(n+2), and V(m)<V(m+1)>V(m+2); or V(0)>V(1)<V(2), V(n)<V(n+1)>V(n+2), and V(m)>V(m+1)<V(m+2). The parameters of the amplitudes V represent a time series, where 0<n<m. These values V fulfill the condition of being values outside of a predetermined noise threshold in order to avoid noise around the baseline 24; specifically, values greater than a noise upper limit threshold 26 or values less than a noise lower limit threshold 25. Also fulfilled is a QRS width condition that m does not exceed a certain value, or an RS width condition that m-n does not exceed a certain value. The QRS width can be divided into a QR width and an RS width which are virtually the same width, a person's normal QRS width is 0.12 s or less, and the QRS width condition is that m be 0.12 or less or the RS width condition is that m-n be 0.06 or less. Therefore, the QRS width condition value and the RS width condition value are respectively about 0.12 s and 0.06 s or less, the preferred maximum values thereof being 0.12 s or 0.06 s.

Of the conditions for retrieving the sampling points previously described, the relationships V(0)<V(1)>V(2), V(n)>V(n+1)<V(n+2), and V(m)<V(m+1)>V(m+2) are referred to as a downward trough condition, while the relationships V(0)>V(1)<V(2), V(n)<V(n+1)>V(n+2), and V(m)>V(m+1)<V(m+2) are referred to as an upward peak condition. With both the downward trough condition and the upward peak condition, V(n+1) is a peak pertaining to an R wave or an S wave, and the peak r′(1) 22 or the peak r′(2) 23 in FIG. 8 represents a position concurrent with the measuring of the point V(n+1). FIGS. 8 and 9 show points detected with the downward trough condition, and if the peak r′(1) 22 in FIG. 8 is a peak pertaining to an R wave, V(n+1) in FIG. 9 is also a peak pertaining to an R wave.

When the R waves or S waves in the electrocardiogram are consistently high, the same condition is consistently fulfilled whether it be an upward peak condition or a downward trough condition, and peaks pertaining to either adjacent R waves or adjacent S waves continue to be detected. However, when the R waves and S waves in the electrocardiogram have approximately the same wave height, the upward peak condition and the downward trough condition irregularly coincide and jittering therefore occurs in the measured values of the heartbeat intervals (an S wave is measured after an R wave without measuring the adjacent R wave, causing discrepancies with the data that was originally to be measured). In view of this, once peaks pertaining to R waves or S waves are detected with the downward trough condition or the upward peak condition, jittering can be avoided by detecting the peaks pertaining to R waves or S waves while fulfilling the downward trough condition or the upward peak condition. Specifically, for example, once peaks pertaining to R waves are detected with the upward peak condition, only peaks pertaining to R waves are thereafter detected.

For example, when three living body information groups are simultaneously transmitted wirelessly and when a peak pertaining to a first R wave is detected to begin with, a peak pertaining to an adjacent second R wave is then detected, after which the temporal interval between the first and second peaks is calculated while a peak pertaining to a third R wave adjacent to the peak pertaining to the second R wave is detected, the temporal interval between the second and third peaks is calculated while a peak pertaining to a fourth R wave adjacent to the peak pertaining to the third R wave is detected, the temporal interval between the third and fourth peaks is calculated, and the temporal intervals between all the peaks and the living body information (triaxial acceleration and temperature) associated with these intervals are wirelessly transmitted together.

Because the heights of the R waves or S waves change due to diurnal fluctuations, the conditions should be reversed if one wave height is consistently higher than the other wave height. For example, the measurements here were taken with the upward peak condition fulfilled, but in a state in which the wave height for the fulfilled downward trough condition is consistently greater than the wave height for the fulfilled upward peak condition, a transition should be made to the downward trough condition (specifically, for example, the wave height of the R wave continues to be observed even in a state in which the upward peak condition is fulfilled and peaks pertaining to S waves continue to be detected).

The wave height value in the region satisfying the retrieved condition is made to be the greater of the following two values: |V(1)−V(n+1)| and |(Vn+1)−V(m+1)|. The heartbeat interval measuring means 4 is configured so that when the ratio Uh/Lh, which is the ratio of the wave height value Uh in the region satisfying the upward peak condition to the wave height value Lh in the region satisfying the downward trough condition, is equal to or less than a threshold Th, the downward trough condition is satisfied; and when the ratio Lh/Uh of the wave height value Lh to the wave height value Uh is equal to or less than the threshold Th, the upward peak condition is satisfied and a switch is made either from peaks pertaining to R waves to peaks pertaining to S waves, or from peaks pertaining to S waves to peaks pertaining to R waves. The threshold Th is preferably 0.4 to 0.7. When the threshold Th is greater than 0.7, there is a possibility that reversal will occur frequently. Reversals are preferably as infrequent as possible because jittering occurs in the instant that a reversal takes place. Reversals do not take place when the threshold Th is less than 0.4.

Because noise such as that of myoelectricity sometimes gets in between one R wave and another adjacent R wave, there is a possibility of the noise being mistakenly detected as a peak pertaining to an R wave or an S wave. Because variations in a person's normal heartbeat intervals are about ±25%, the time when the next (adjacent) peak pertaining to an R wave or an S wave will appear can be predicted to some extent. Therefore, once a peak pertaining to an R wave or an S wave is detected, if the condition is fulfilled that a peak pertaining to an R wave or an S wave not be detected within a certain time duration thereafter, it is possible to minimize mistaken detections of peaks pertaining to R waves or S waves due to noise such as that of myoelectricity. It is also possible to further minimize mistaken detections by applying the previously described low-pass filter. Because the maximum human heart rate is about 200 beats per minute, the time duration for not detecting a peak pertaining to an R wave or an S wave is preferably 0.3 s or less, or about 50% of the heartbeat interval measured immediately prior.

Because the present example is configured as described above, when electrodes are brought in contact with a living body, a change in the voltage obtained from the electrodes is electrically processed to create an electrocardiogram signal, and from this electrocardiogram signal, a heartbeat interval is measured from the interval between one R wave and another adjacent R wave or the interval between one S wave and another adjacent S wave within the electrocardiogram signal; the heartbeat interval can be measured by an extremely simple data process using a microcomputer or the like, and it is possible to store the heartbeat interval measurement results alone without storing waveform data of the electrocardiogram.

Therefore, the present example is a biological information measuring apparatus in which a small-capacity memory can be used, the apparatus can be reduced in size accordingly, the wearer can therefore be minimally inconvenienced, the life of the battery can be extended, and the apparatus can be satisfactorily used while worn continuously for a long period of time.

1. A biological information measuring apparatus capable of measuring at least a heartbeat interval of a living body and capable of being attached to a living body, the biological information measuring apparatus characterized in comprising: a plurality of electrodes that come in contact with the living body; signal processing means for electrically processing a change in voltage obtained from the electrodes and creating an electrocardiogram signal; heartbeat interval measuring means for measuring, from the electrocardiogram signal, a heartbeat interval from an interval between one R wave and another adjacent R wave or an interval between one S wave and another adjacent S wave in the electrocardiogram signal; triaxial acceleration measuring means; and temperature measuring means; the biological information measuring apparatus further comprising wireless transmission means for simultaneously and wirelessly transmitting the heartbeat interval obtained by the heartbeat interval measuring means, the triaxial acceleration obtained by the triaxial acceleration measuring means, and the temperature obtained by the temperature measuring means.

2. The biological information measuring apparatus according to claim 1, characterized in being configured so that a high-pass filter is provided to the signal processing means, the heartbeat interval measuring means isochronally samples the electrocardiogram signals passing through the high-pass filter, peaks pertaining to R waves or S waves are detected at two points using the method described below, and a heartbeat interval is measured using an interval between peaks pertaining to the R waves or S waves either as an interval between one R wave and another adjacent R wave, or as an interval between one S wave and another adjacent S wave:
A region is retrieved where adjacent amplitudes V satisfy the relationships for a downward trough condition: V(0)<V(1)>V(2), V(n)>V(n+1)<V(n+2), and V(m)<V(m+1)>V(m+2), or the relationships for an upward peak condition: V(0)>V(1)<V(2), V(n)<V(n+1)>V(n+2), and V(m)>V(m+1)<V(m+2); and V(n+1) in the region satisfying the downward trough condition or the upward peak condition is detected as a peak pertaining to an R wave or a peak pertaining to an S wave; the V parameters representing a time series, where 0<n<m; and the amplitudes V being values outside of a predetermined noise threshold.

3. The biological information measuring apparatus according to claim 1, characterized in being configured so that that a high-pass filter is provided to the signal processing means, the heartbeat interval measuring means isochronally samples the electrocardiogram signals passing through the high-pass filter, peaks pertaining to R waves or S waves are detected at two points using a method described below, and a heartbeat interval is measured using an interval between peaks pertaining to R waves or S waves either as an interval between one R wave and another adjacent R wave, or as an interval between one S wave and another adjacent S wave:
(1) A region is retrieved where adjacent amplitudes V satisfy the relationships for a downward trough condition: V(0)<V(1)>V(2), V(n)>V(n+1)<V(n+2), and V(m)<V(m+1)>V(m+2), or the relationships for an upward peak condition: V(0)>V(1)<V(2), V(n)<V(n+1)>V(n+2), and V(m)>V(m+1)<V(m+2); and V(n+1) in the region satisfying the downward trough condition or the upward peak condition is detected as a peak pertaining to an R wave or a peak pertaining to an S wave; the V parameters representing a time series, where 0<n<m; and the amplitudes V being values outside of a predetermined noise threshold.
(2) After any peak fulfilling the downward trough condition or the upward peak condition and pertaining to an R wave or an S wave has been detected, a peak fulfilling the same condition and pertaining to an adjacent R wave or S wave is detected.

4. The biological information measuring apparatus according to claim 2, characterized in that the heartbeat interval measuring means either sets m to 0.12 seconds or less or sets m-n to 0.06 seconds or less.

5. The biological information measuring apparatus according to claim 2, characterized in that the heartbeat interval measuring means is configured so that: respective wave height values {whichever is greater between |V(1)−V(n+1)| and |V(n+1)−V(m+1)|} in the retrieved regions satisfying the conditions are compared; the downward trough condition is fulfilled when a ratio Uh/Lh, which is a ratio of a wave height value Uh in the region satisfying the upward peak condition to a wave height value Lh in the region satisfying the downward trough condition, is equal to or less than a threshold Th; and the upward peak condition is fulfilled when a ratio Lh/Uh, which is a ratio of the wave height value Lh to the wave height value Uh, is equal to or less than a threshold Th.

6. The biological information measuring apparatus according to claim 5, characterized in that the threshold Th is 0.4 to 0.7.

7. The biological information measuring apparatus according to claim 6, characterized in that the heartbeat interval measuring means is configured so that once a peak pertaining to either an R wave or an S wave has been detected, a peak pertaining to the next R wave or S wave is not detected within a range of 0.3 second or less.

8. The biological information measuring apparatus according to claim 2, characterized in that the high-pass filter only lets through frequencies of at least a predetermined threshold set in a range of 8 Hz to 30 Hz.

9. The biological information measuring apparatus according to claim 8, characterized in that a low-pass filter is provided to the signal processing means.

10. The biological information measuring apparatus according to claim 9 characterized in that the low-pass filter lets through only frequencies at or below a predetermined threshold set to a value greater than 30 Hz.

11. The biological information measuring apparatus according to claim 2, characterized in comprising wireless transmission means for grouping the heartbeat interval obtained by the heartbeat interval measuring means, the triaxial acceleration obtained by the triaxial acceleration measuring means, and the temperature obtained by the temperature measuring means as a single living body information group, and wirelessly transmitting one to ten living body information groups simultaneously to a recording device.

12. The biological information measuring apparatus according to claim 11, characterized in that the triaxial acceleration measuring means is configured so as to measure the maximum value or average value of the absolute values of acceleration in each of three axes either between one R wave and another adjacent R wave or between one S wave and another adjacent S wave in the electrocardiogram signal obtained by the signal processing means.

13. The biological information measuring apparatus according to claim 11, characterized in that the triaxial acceleration measuring means is configured so as to measure the maximum value and minimum value of acceleration in each of three axes either between one R wave and another adjacent R wave or between one S wave and another adjacent S wave in the electrocardiogram signal obtained by the signal processing means.

14. The biological information measuring apparatus according to claim 12, characterized in comprising recording means for internally recording heartbeat interval, triaxial acceleration, and temperature.

15. The biological information measuring apparatus according to claim 13, characterized in comprising recording means for internally recording heartbeat interval, triaxial acceleration, and temperature.
